(19) United States
(12) Patent Application Publication
(10) Pub. No.: US 2008/0280866 A1
(43) Pub. Date: Nov. 13, 2008

(54) Method of Treating or Sepsis

(76) Inventor: Carl J. Hauser, Mountainside, NJ (US)

Correspondence Address: Moser IP Law Group, 1030 BROAD STREET, 2ND FLOOR, SHREWSBURY, NJ, 07702, US

(21) Appl. No.: 11/658,116

(22) PCT Filed: Jul. 7, 2005
(86) PCT No.: PCT/US05/24074

§ 371 Date: Jan. 22, 2007

Related U.S. Application Data
(60) Provisional application No. 60/589,633, filed on Jul. 21, 2004.

(51) Int. Cl. A61K 31/555; A61K 31/425; A61P 29/00

(52) U.S. Cl. 514/186; 514/370

(57) ABSTRACT

Provided is a method of treating trauma or sepsis comprising: administering an effective amount of one or both of a SOC channel-selective inhibitor or a SphK inhibitor to an animal at risk of inflammation-mediated organ damage from trauma or sepsis.

The present invention relates to treating or ameliorating trauma or sepsis.

Trauma causes over 150,000 deaths per year in the USA. It is the leading cause of death between the ages of 1 and 45 and between the ages of 1 and 36, deaths from trauma far exceed all other deaths combined (1, 2). Over 50,000 preventable deaths occur each year when trauma and hemorrhagic shock (“T/HS”) trigger the systemic inflammatory response syndrome (“SIRS”) (3). SIRS initiates neutrophil (“PMN”) dysfunction (4-6), and PMN dysfunction triggers acute respiratory distress syndrome (“ARDS”), multiple organ failure syndrome (“MOF”) or sepsis (7-13). Moreover ARDS, MOF and sepsis are the most frequent treatable causes of prolonged critical illness, ICU and ventilator utilization, and death after injury. In 1977, about 300,000 persons developed acute respiratory distress syndrome from all causes. The disease specific mortality rate was 40% and rose to 60-90% when combined with the failure of other organs (14). Although modern ICU care has lowered the mortality rate of acute respiratory distress syndrome to about 30%, its incidence has remained stable (15). Thus PMN inflammation after trauma is a huge public health problem in terms of morbidity, mortality, and health care costs.

Neutrophil-mediated inflammation plays a key role in trauma, shock, organ failure and sepsis (7-11, 4). PMN are primed and activated by trauma and shock. This contributes to end-organ leukosequestration, acute lung injury (“ALI”) and eventually to acute respiratory distress syndrome or multiple organ failure syndrome (MOF). Paradoxically, PMN dysfunction after trauma and shock can also predispose patients to sepsis (16-22). The molecular mechanisms leading to PMN dysfunction, acute respiratory distress syndrome, multiple organ failure syndrome and sepsis after trauma and shock are incompletely understood, but it is known that a wide variety of G-protein coupled inflammatory chemoattractants are involved in PMN activation. G-protein coupled receptor agonists attract and activate PMN through a superfamily of 7-transmembrane domain receptors. These receptors effect outside-in signaling via hetero-trimeric G-proteins which activate PMN in great measure by mobilizing cell calcium. Agonist-initiated Ca2+ mobilization is believed to be critical to PMN activation, regulating respiratory burst, degranulation, motility, gene expression, and apoptosis (23-26).

After injury and shock, a complex inflammatory process leads from PMN activation to end-organ attack and clinical organ failure. An overwhelming body of work in the published literature suggests that mediators released during systemic inflammatory response syndrome activate PMN (3), that acute respiratory distress syndrome occurs when pathologically activated PMN attack micro-vascular endothelial cells (reviewed by Abraham (27), Hogg (9), Pober (28), Partrick (10) and Luster (29)), and that acute respiratory distress syndrome does not occur in the absence of PMN (12). Thus this area has been the subject of an enormous amount of study and much is known, but many elements in this sequence of events remain unclear.

Sphingosine-1 phosphate (“S1P”) is believed to be a crucial mediator of PMN calcium entry via store-operated calcium channels (“SOC”) (30). Calcium entry is a pleuripotent activator of PMN functions. PMN Ca²⁺ entry responses to S1P are modified by trauma. Data herein shows that (i) the inhibition of sphingosine kinase (“sphK”) (which enzyme produces S1P) or (ii) the inhibition of PMN Ca2+ entry in response to S1P can prevent lung injury due to traumatic shock. Surprisingly, these advantages arise without apparent vascular or other complications that might be expected from other interventions in calcium-mediated pathways. It is believed that this is due to S1P-mediated calcium entry pathways not being important for the calcium pathways involved in hemodynamic regulation, and similarly that available SOC blocking agents are relatively ineffective in blocking the L-type calcium channels involved in hemodynamic regulation.

Sepsis, independent of trauma, appears to generate many of the same neutrophil mediated injuries described above (31). Thus, the treatment provided here is believed to beneficially contribute to the treatment of sepsis.

### SUMMARY OF THE INVENTION

In one embodiment, the invention provides a method of treating trauma or sepsis comprising: administering an effective amount of one or both of a SOC channel-selective inhibitor or a SphK inhibitor to an animal at risk of inflammation-mediated organ damage from trauma or sepsis.

In one embodiment, when treating sepsis, the method further comprises administering an effective amount of an antimicrobial agent in a dosage regimen effective to provide a clinical improvement in the sepsis and thereafter beginning the administering one or both of a SOC channel-selective inhibitor or a SphK inhibitor.

### BRIEF DESCRIPTION OF THE DRAWINGS

FIG. 1 shows the effect of 4-[4-(4-chloro-phenyl)-thiazol-2-ylamino]-phenol (“SKI-2”) treatment on PMN infiltration into the lung (MPO) and on subsequent lung injury (Evans Blue leak).

FIG. 2 shows the effect of 4-[4-(4-chloro-phenyl)-thiazol-2-ylamino]-phenol or N-propargyl-nitrendipine on PMN expression of the adhesion molecule CD11b when administered after trauma.

FIG. 3 shows that 4-[4-(4-chloro-phenyl)-thiazol-2-ylamino]-phenol or N-propargyl-nitrendipine given in post-treatment decreased the priming of PMN for production of toxic oxygen free radical species.

FIG. 4 shows the effect of 4-[4-(4-chloro-phenyl)-thiazol-2-ylamino]-phenol on PMN phosphorylation of p38 MAP kinase in response to trauma and hemorrhagic shock.

### DETAILED DESCRIPTION OF THE INVENTION

### SphK Inhibitors

Inhibitors of SphK are known, as illustrated in Smith et al., WO03/105840. Additional candidate inhibitors can be assayed by methods described in WO03/105840 or otherwise known in the art. The teachings of WO03/105840 are hereby incorporated by reference herein in their entirety. The target used in the assay can be the predominant SphK in an animal, but is preferable type 1 sphK from human. See, e.g., Melendez et al., Gene 251:19-26 (2000). Compounds described in WO03/105840 include:
1. 5-(2,4-Dihydroxy-benzylidene)-3-(4-methoxy-phenyl)-2-thioxo-thiazolidin-4-one;
2. 5-Naphthalen-2-yl-2H-pyrazole-3-carboxylic acid (2-hydroxy-naphthalen-1-ylmethylene)-hydrazide;
3. 4-[4-(4-Chloro-phenyl)-thiazol-2-ylamino]-phenol (SKI-2);
4. 2-(3,4-Dihydroxy-benzylidene)-benzo[b]thiophen-3-one;
5. 2-(3,4-Dihydroxy-benzylidene)-benzofuran-3-one;
6. N-Benzothiazol-2-yl-2-[4-hydroxy-1-(4-methoxy-phenyl)-6-oxo-1,6-dihydro-pyrimidin-2-ylsulfanyl]-acetamide;
7. 2-(1-adamantyl)-4-[(4-oxidophenyl)sulfonyl]benzenolate disodium salt;
8. 2-(2-Hydroxy-3,5-diiodo-benzylidene)-6,7-dimethyl-benzo[4,5]imidazo[2,1-b]thiazol-3-one;
9. 2-(4-Benzo[1,3]dioxol-5-yl-thiazol-2-yl)-3-(3,4-dihydroxy-phenyl)-acrylonitrile;
10. 4-(7-Ethoxy-4,4-dimethyl-4,5-dihydro-2,3-dithia-5-aza-cyclopenta[α]naphthalen-1-ylideneamino)-phenol;
11. 3,4-Dihydroxy-1,6-diphenyl-hexa-2,4-diene-1,6-dione;
12. the following compound:

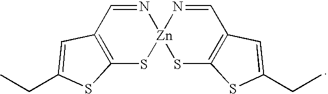

Formulas reported in WO03/105840 as defining sphK inhibitors include those outlined below.

One class of inhibitors comprises compounds of formula (I):

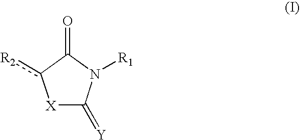

and pharmaceutically acceptable salts thereof, wherein
X is CHR3 or S;
Y is O or S;
R1 and R2 are independently H, (C₁-C₁₅)alkyl, (C₃-C₇)cycloalkyl, —C₁-C6-alkyl-(C₃-C₇)cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₃, —OC(O)R₃, mono or dialkylcarbamoyl, mono or dialkylamino, aminoalkyl, mono- or dialkylaminoalkyl, thiocarbamoyl, or mono or dialkylthiocarbamoyl; and
R₃ is H, alkyl, aryl, arylalkyl, cycloalkyl, cycloalkylalkyl, heteroaryl, heteroarylalkyl, heterocycloalkyl, or heterocycloalkylalkyl;
   wherein the alkyl and ring portion of each of the above substituents is optionally substituted with up to 5 groups that are independently (C₁-C₆)allyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆)alkyl.

The invention also provides compounds of formula II:

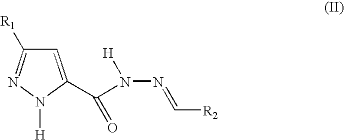

and pharmaceutically acceptable salts thereof, wherein
R₁ and R₂ are independently H, (C₁-C₁₅)alkyl, (C₃-C₇)cycloalkyl, —C₁-C₆-alkyl-(C₃-C₇)cycloalkyl, aryl, —C₁-C₆-alkyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆ alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₈, —OC(O)R₈, mono or dialkylcarbamoyl, mono or dialkylamino, aminoalkyl, mono- or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl;
      wherein the alkyl and ring portion of each of the above substituents is optionally substituted with up to 5 groups that are independently (C₁-C₆)alkyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆)alkyl.

The invention further provides compounds of formula (III):

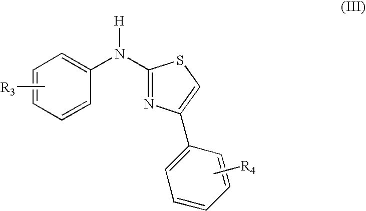

and pharmaceutically acceptable salts thereof, wherein
R₃ and R₄ are independently H, (C₁-C₁₅)alkyl, (C₃-C₇)cycloalkyl, —C₁-C₆-alkyl-(C₃-C₇) cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₈, —OC(O)R₈, mono or dialkylcarbamoyl, mono or dialkylamino, aminoalkyl, mono- or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl,
      wherein the alkyl and ring portion of each of the above substituents is optionally substituted with up to 5 groups that are independently (C₁-C₆) alkyl, halogen, haloalkyl, —CF₃, —OCF₃, —OH, C₁-C₆ alkoxy, hydroxyalkyl, —CN, —CO₂H, —SH, —S-alkyl, —NO₂, or NR′R″, wherein R′ and R″ are independently H or (C₁-C₆) alkyl.

The invention further provides compounds of formula (IV):

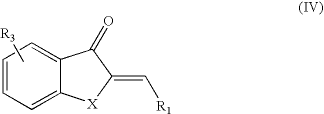

and pharmaceutically acceptable salts thereof, wherein
X is O or S;
R₁ and R₃ are independently H, (C₁-C₁₅)alkyl, (C₃-C₇)cycloalkyl, —C₁-C₆-alkyl-(C₃-C₇) cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₉, —OC (O)R₈, mono or dialkylcarbamoyl, mono or dialkylamino, aminoalkyl, mono- or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl
      wherein the alkyl and ring portion of each of the above substituents is optionally substituted with up to 5 groups that are independently (C₁-C₆) alkyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆)alkyl.

The invention further provides compounds of formula (V):

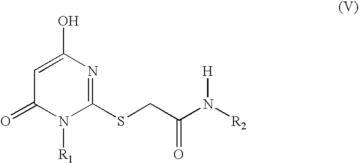

and pharmaceutically acceptable salts thereof, wherein
R1 and R2 are independently H, (C₁-C₁₅)alkyl, (C₃-C₇)cycloalkyl, —C₁-C₆-alkyl-(C₃-C₇) cycloalkyl, aryl, —C₁-C₆-alkyl-aryl, —C₂-C₆-alkenyl-aryl, heteroaryl, —C₁-C₆ alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₈, —OC(O)R₈, mono or dialkylcarbamoyl, mono or dialkylamino, aminoalkyl, mono- or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl;
      wherein the alkyl and ring portion of each of the above substituents is optionally substituted with up to 5 groups that are independently (C₁-C₆) alkyl, halogen, haloalkyl, —CF₃, —OCF₃, —OH, C₁-C₆ alkoxy, hydroxyalkyl, —CN, —CO₂H, —SH, —S-alkyl, —NO₂, or NR′R″, wherein R′ and R″ are independently H or (C₁-C₆) alkyl.

The invention further provides compounds of formula (VI):

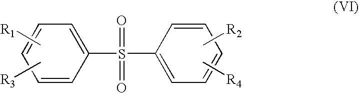

and pharmaceutically acceptable salts thereof,
wherein R₁, R₂, R₃, and R₄ are independently H, (C₁-C₁₅)alkyl, (C₃-C₇)cycloalkyl, —C₁-C₆-alkyl-(C₃-C₇)cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₈, —OC(O)R, mono or dialkylcarbamoyl, mono or dialkylamino, aminoalkyl, mono or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl;
      wherein the alkyl and ring portion of each of the above substituents is optionally substituted with up to 5 groups that are independently (C₁-C₆)-alkyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆) alkyl.

The invention also provides compounds of formula (VII):

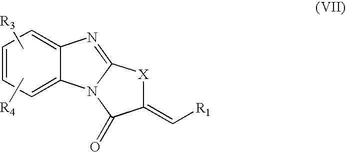

and pharmaceutically acceptable salts thereof,
wherein X is O or S;
R₁, R₃ and R₄ are independently H, (C₁-C₁₅)alkyl, (C₃-C₇)cycloalkyl, —C₁-C₆-alkyl-(C₃-C₇) cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₈, —OC(O)R, mono or dialkylcarbarnoyl, mono or dialkylamino, aminoalkyl, mono- or dialkylaminoalkyl, thiocarbamoyl, or mono or dialkylthiocarbamoyl; and
   Ra is H, alkyl, aryl, arylalkyl, cycloalkyl, cycloalkylalkyl, heteroaryl, heteroarylalkyl, heterocycloalkyl, or heterocycloalkylalkyl;
      wherein the alkyl and ring portion of each of the above substituents is optionally substituted with up to 5 groups that are independently (C₁-C₆)alkyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆) alkyl.

The invention also provides compounds of formula (VIII):

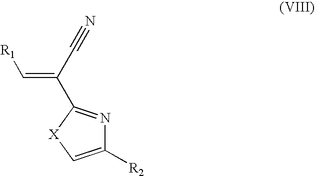

and pharmaceutically acceptable salts thereof, wherein
X is O or S;
R₁, and R₂ are independently H, (C₁-C₁₅) alkyl, (C₃-C₇)cycloalkyl, —C₁-C₆-alkyl-(C₃-C₇) cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂Re, —OC(O)R₈, mono or dialkylcarbamoyl, mono or dialkylamino, aminoalkyl, mono- or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl;
      wherein the alkyl and ring portion of each of the above substituents is optionally substituted with up to 5 groups that are independently (C₁-C₆)alkyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆)alkyl.

The invention also provides compounds of formula (IX):

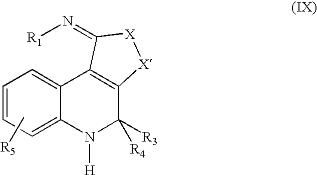

and pharmaceutically acceptable salts thereof, wherein
X and X′ are independently O or S;
R₁, R₃, R₄ and R₅ are independently H, (C₁-C₁₅)alkyl, (C₃-C₇)cycloalkyl, —C₁-C₆-alkyl-(C₃-C₇) cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₈, —OC(O)R₈, mono or dialkylcarbamoyl, mono or dialkylamino, aminoalkyl, mono- or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl,
      wherein the alkyl and ring portion of each of the above substituents is optionally substituted with up to 5 groups that are independently (C₁-C₆) alkyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆) alkyl.

The invention also provides compounds of formula (X):

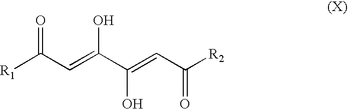

and pharmaceutically acceptable salts thereof, wherein
R₁ and R₂ are independently H, (C₁-C₁₅) alkyl, (C₃-C₇)cycloalkyl, —C₁-C₆-alkyl-(C₃-C₇) cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₈, —OC(O)R₈, mono or dialkylcarbarnoyl, mono or dialkylamino, aminoalkyl, mono- or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl;
      wherein the alkyl and ring portion of each of the above substituents is optionally substituted with up to 5 groups that are independently (C₁-C₆) alkyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆) alkyl.

The invention also provides compounds of formula (XI):

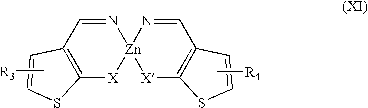

and pharmaceutically acceptable salts thereof, wherein
X and X′ are independently O or S;
R₃ and R₄ are independently H, (C₁-C₁₅)alkyl, (C₃-C₇)cycloalkyl, —C₁-C₆-alkyl-(C₃-C₇) cycloalkyl, aryl, —C₁-C₆-alkyl-aryl, —C₂-C₆-alkenyl-aryl, heteroaryl, —C₁-C₆-alkyl-heteroaryl, heterocycloalkyl, —C₁-C₆-alkyl-heterocycloalkyl, halogen, haloalkyl, —OH, C₁-C₆-alkoxy, hydroxyalkyl, alkanoyl, —COOH, carbamoyl, mono or dialkylaminocarbamoyl, —SH, —S-alkyl, —CF₃, —OCF₃, —NO₂, —NH₂, —CO₂R₈, —OC(O)RB, mono or dialkylcarbamoyl, mono or dialkylamino, aminoalkyl, mono- or dialkylaminoalkyl, thiocarbamoyl, or mono or dialkylthiocarbamoyl; and
   R₈ is H, alkyl, aryl, arylalkyl, cycloalkyl, cycloalkylalkyl, heteroaryl, heteroarylalkyl, heterocycloalkyl, or heterocycloalkylalkyl;
      wherein the alkyl and ring portion of each of the above substituents is optionally substituted with up to 5 groups that are independently (C₁-C₆)alkyl, halogen, haloalkyl, —CF₃, —OCF₃, —OH, C₁-C₆-alkoxy, hydroxyalkyl, —CN, —CO₂H, —SH, —S-alkyl, —NO₂, or NR′R″, wherein R′ and R″ are independently H or (C₁-C₆) alkyl.

Where not specified, alkyl and alkoxy are C₁-C₆, carbamoyl and alkanoyl moieties are C₂-C₆, and cycloalkyls are C₃-C₇.

### SOC Channel-Selective Inhibitors

The in vivo results described below were obtained with a SOC channel inhibitor having potency in cell culture assays in the micromolar range. Thus, very high potency is not required. What is believed to be needed is potency of the same order of magnitude or lower than any overlapping potency as an inhibitor of voltage-dependent L-type calcium channels. Cross-potency beyond this level is believed create too great a risk of unwanted hemodynamic effects. In some embodiments, the potency for SOC channel inhibition is higher than the potency for voltage-dependent L-type calcium channels.

A number of agents meeting these criteria have been described by Harper et al., Biochemical Pharmacology 65: 329-38, 2003 (“Harper”), the disclosures of which are hereby incorporated by reference herein in their entirety. Such compounds include 1,4-dihydropyridines substituted C2 and C6 with methyl and at the remaining ring positions as follows:

| | N1 | C3 | C4 | C5 |
|---|---|---|---|---|
| 30. | Me | COOMe | 3-NO₂-Ph | COOMe |
| 32. | Me | COOEt | 3-NO₂-Ph | COOMe |
| 33. | Et | COOEt | 3-NO₂-Ph | COOMe |
| 34. | Allyl | COOEt | 3-NO₂-Ph | COOMe |
| 35. | Propargyl | COOEt | 3-NO₂-Ph | COOMe |

Compound 35 is also known as MRS 1845 and N-propargyl-nitrendipine.

These or other compounds can be tested for inhibition of SOC channels using cells loaded with calcium sensitive dye as described in Harper, or by other methods known in the art. As in Harper, the dye can be Fluo3-Am, fluo4-AM or fura2-AM as available from Molecular Probes, Inc., Eugene, Oreg. The cells used for testing inhibition of SOC channels can be, for example, PMN cells or, as in Harper, HL-60 cells. The cells used to measure inhibition of voltage-dependent L-type calcium channels can be, for example, GH₄C₁ cells (as used in Harper). With such tests Harper determined the IC₅₀ values (in micromolar units) for the above-listed compounds as follows:

| Cmpd | SOC IC₅₀ | L-Type IC₅₀ |
|---|---|---|
| 30 | 2.8 ± .03 | 2 |
| 32 | 2.6 ± 1.1 | 1.3 ± 0.3 |
| 33 | 6 | 2.3 ± 0.4 |
| 34 | 6.8 ± 1.8 | 3.8 ± 1.8 |
| 35 | 1.7 ± 1.3 | 2.1 ± 0.2 |

Potential inhibitors of SOC channels are preferably titrated to determine any concentration at which they may cause an increase in intracellular calcium. Preferably, such concentrations are greater than the concentrations for SOC channel inhibition by 5-fold, 6-fold, 7-fold, 8-fold, 9-fold, 10-fold or higher.

Compounds used may be, in certain embodiments, dihydropyridines that are N-substituted. Or, dihydropyridines that are N-substituted with (i) C₁₋₆-alkyl, C₂₋₆-alkenyl or C₂₋₆-alkynyl, which (of the foregoing) may optionally be substituted with one or more substituents selected from halogen, —CN, —CF₃, —OCHF₂, —OCF₃, —NO₂, —OR¹⁷ and —NR¹⁷R¹⁸, (ii) —CH₂CN, —CHF₂ or —CF₃, or (iii) aryl-C₁₋₃-alkyl or heteroaryl-C₁₋₃-alkyl, which (of the foregoing) may optionally be substituted with one or more substituents selected from halogen, —C(O)OR¹⁷, —CN, —CF₃, —OCF₃, —OCHF₂, —NO₂, —OR¹⁷, —NR¹⁷R¹⁸ and C₁₋₆-alkyl, where R¹⁷ and R¹⁸ are independently hydrogen or C₁₋₃-alkyl. In another embodiment, the compounds may be according to Formula (XII), as follows:

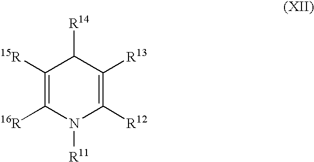

In formula XII:
R¹¹ is (i) —CH₂CN, —CHF₂ or —CF₃, or (ii) C₁₋₆-alkyl, C₂₋₆-alkenyl or C₂₋₆-alkynyl, which may optionally be substituted with one or more substituents selected from halogen, —CN, —CF₃, —OCHF₂, —OCF₃, —NO₂, —OR¹⁷ and —NR¹⁷R¹⁸, or (iii) aryl-C₁₋₃-alkyl or heteroaryl-C₁₋₃-alkyl, which may optionally be substituted with one or more substituents selected from halogen, —C(O)OR¹⁷, —CN, —CF₃, —OCF₃, —OCHF₂, —NO₂, —OR¹⁷, —NR¹⁷R¹⁸ and C₁₋₆-alkyl, where R¹⁷ and R¹⁸ are independently hydrogen or C₁₋₃-alkyl;
R¹² and R¹⁶ are independently (i) hydrogen, halogen, —CN, —CH₂CN, —CHF₂, —CF₃ or (iii) C₁₋₆-alkyl, C₂₋₆-alkenyl or C₂₋₆-alkynyl, which may optionally be substituted with one or more substituents selected from halogen, —CN, —CF₃, —OCHF₂, —OCF₃, —NO₂, —OR¹⁷ and —NR¹⁷R¹⁸;
R¹³ and R¹⁵ are independently (i) hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, —OR¹⁹, —NR¹⁹R²⁰, —SR¹⁹, —NR¹⁹S(O)₂R²⁰, —S(O)₂NR¹⁹R²⁰, —S(O)NR¹⁹R²⁰, —S(O)R¹⁹, —S(O)₂R¹⁹, —C(O)NR¹⁹R²⁰, —OC(O)NR¹⁹R²⁰, —NR¹⁹C(O)R²⁰, —CH₂C(O)NR¹⁹R²⁰, —OCH₂C(O)NR¹⁹R²⁰, —CH₂OR¹⁹, —CH₂NR¹⁹R²⁰, —OC(O)R²², —C(O)R²² or —C(O)OR²² (ii) C₁₋₆-alkyl, C₂₋₆-alkenyl or C₂₋₆-alkynyl, which may optionally be substituted with one or more substituents selected from halogen, —CN, —CF₃, —OCHF₂, —OCF₃, —NO₂, —OR¹⁹ and —NR¹⁹R²⁰, wherein R¹⁹ and R²⁰ are independently hydrogen, C₁₋₆-alkyl, aryl-C₁₋₆-alkyl or aryl, or R¹⁹ and R²⁰ when attached to the same nitrogen atom together with the nitrogen atom may form a 3 to 8 membered heterocyclic ring optionally containing one or two further heteroatoms selected from nitrogen, oxygen and sulfur, and optionally containing one or two double bonds, wherein the aryls of R¹⁹ or R²⁰ may optionally be substituted with one or more substituents selected from halogen, —C(O)OR¹⁹*, —CN, CF₃, —OCF₃, —OCHF₂, —NO₂, —OR^{19*}, —NR¹⁹*R²⁰* and C₁₋₆-alkyl, wherein R¹⁹* and R²⁰* are independently hydrogen or C₁₋₆-alkyl; and
R¹⁴ is (i) hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, —OR¹⁹, —NR¹⁹R²⁰, —SR¹⁹, —NR¹⁹S(O)₂R²⁰, —S(O)₂NR¹⁹R²⁰, —S(O)NR¹⁹R²⁰, —S(O)R¹⁹, —S(O)₂R¹⁹, —C(O)NR¹⁹R²⁰, —OC(O)NR¹⁹R²⁰, NR¹⁹C(O)R²⁰, —CH₂C(O)NR¹⁹R²⁰, —OCH₂C(O)NR¹⁹R²⁰, —CH₂OR¹⁹, —CH₂NR¹⁹R²⁰, —OC(O)R¹⁹, —C(O)R¹⁹ or —C(O)OR¹⁹ (ii) C₁₋₆-alkyl, C₂₋₆-alkenyl or C₂₋₃-alkynyl, which may optionally be substituted with one or more substituents selected from halogen, —CN, —CF₃, —OCHF₂, —OCF₃, —NO₂, —OR¹⁹ and —NR¹⁹R²⁰, or (iii) aryl, heteroaryl, aryl-C₁₋₃-alkyl or heteroaryl-C₁₋₃-alkyl, which may optionally be substituted with one or more substituents selected from halogen, —C(O)OR¹⁹, —CN, —CF₃, —OCF₃, —OCHF₂, —NO₂, —OR¹⁹, —NR¹⁹R²⁰, C₁₋₆-alkyl, C₁₋₆-alkenyl and C₁₋₆-alkynyl.

In certain embodiments, one or more recitations of C₁₋₆ or C₂₋₆ can be replaced with recitations of C₁₋₃ or C₂₋₃.

Other SOC channel inhibitors are known in the art and can be used in the treatment of the invention. For example, the lanthanide gadolinium (Gd3+), which can be administered as an appropriate salt, inhibits PMN SOC at low-mid nanomolar concentrations. The appropriate amounts for the use of the invention can be titrated based on inhibition data using PMN, which PMN have at least two SOC channels (Itagaki and Hauser, J. Immunol. 168(8):4063-9 (2002). Safe parameters for using Gd3+ have been established by its use in numerous medical imaging applications.

### General Compound Features

The following is a detailed definition of the terms used to describe the compounds used in the invention:

“Halogen” designates an atom selected from the group consisting of F, Cl, Br and I.

The term “C₁₋₆-alkyl” as used herein represents a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like. For this and other recitations of carbon ranges, the range will correspondingly encompass all structural combinations. For this and other recitations of carbon ranges, C₁₋₆ or C₁₋₅ or C₁₋₄ or C₁₋₃ or C₂₋₆ or C₂₋₅ or C₂₋₄ or C₂₋₃ may in some embodiments be substituted.

The term “C₂₋₆-alkenyl” as used herein represents a branched or straight hydrocarbon group having from 2 to 6 carbon atoms and at least one double bond. Examples of such groups include, but are not limited to, vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “C₂₋₆-alkynyl” as used herein represents a branched or straight hydrocarbon group having from 2 to 6 carbon atoms and at least one triple bond. Examples of such groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 2,4-hexadiynyl and the like.

The term “heterocyclyl” as used herein represents a non-aromatic 3 to 10 membered ring containing one or more heteroatoms selected from nitrogen, oxygen and sulfur and optionally containing one or two double bonds. Representative examples are pyrrolidinyl, piperidyl, piperazinyl, morpholinyl, thiomorpholinyl, aziridinyl, tetrahydrofuranyl and the like.

The term “aryl” as used herein is intended to include carbocyclic, aromatic ring systems such as 6 membered monocyclic and 9 to 14 membered bi- and tricyclic, carbocyclic, aromatic ring systems. Representative examples are phenyl, biphenyl, naphthyl, anthracenyl, phenanthrenyl, fluorenyl, indenyl, azulenyl and the like. Aryl is also intended to include the partially hydrogenated derivatives of the ring systems enumerated above. Non-limiting examples of such partially hydrogenated derivatives are 1,2,3,4-tetrahydronaphthyl, 1,4-dihydronaphthyl, indanyl and the like.

The term “heteroaryl” as used herein is intended to include aromatic, heterocyclic ring systems containing one or more heteroatoms selected from nitrogen, oxygen and sulfur such as 5 to 7 membered monocyclic and 8 to 14 membered bi- and tricyclic aromatic, heterocyclic ring systems containing one or more heteroatoms selected from nitrogen, oxygen and sulfur. Representative examples are furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the ring systems enumerated above. Non-limiting examples of such partially hydrogenated derivatives, are 2,3-dihydrobenzofuranyl, pyrrolinyl, pyrazolinyl, indolinyl, oxazolidinyl, oxazolinyl, oxazepinyl and the like.

The term “optionally substituted” as used herein means that the groups in question are either unsubstituted or substituted with one or more of the substituents specified. When the groups in question are substituted with more than one substituent the substituents may be the same or different.

Certain of the above defined terms may occur more than once in the structural formulae, and upon such occurrence each term shall be defined independently of the other.

Furthermore, when using the terms “independently are” and “independently selected from” it should be understood that the groups in question may be the same or different.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

Also intended as pharmaceutically acceptable acid addition salts are the hydrates, which the present compounds, are able to form. Furthermore, the pharmaceutically acceptable salts comprise basic amino acid salts such as lysine, arginine and ornithine. The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses use of prodrugs and active metabolites of the present compounds. Prodrugs on administration undergo chemical conversion by metabolic processes before becoming pharmacologically active substances. In general, such prodrugs will be functional derivatives of then compounds of the general formula (I), which are readily convertible in vivo into the required compound of the formula (I). Conventional procedures for the selection and preparation of suitable prodrug derivatives are described, for example, in “Design of Prodrugs”, ed. H. Bundgaard, Elsevier, 1985.

Throughout the specification, groups and substituents thereof may be chosen to provide stable moieties and compounds.

### Treatment Parameters

The present invention provides methods and compositions that can be used advantageously to prevent or attenuate late complications that trauma or sepsis patients may experience subsequent to their injury and/or as a result of medical interventions that may be used to treat their injuries. The methods are carried out by administering to a trauma patient one or both of a SOC channel-selective inhibitor or a SphK inhibitor, in a manner that is effective for preventing or attenuating one or more late complications of trauma. A manner effective for preventing or attenuating late complications may comprise administering a predetermined amount of one or both of a SOC channel-selective inhibitor or a SphK inhibitor, and/or utilizing a particular dosage regimen, formulation, mode of administration, and the like. The efficacy of the methods of the invention in preventing late complications of trauma may be assessed using one or more conventionally used parameters of late complications (see below). Late complications that may be prevented by the methods of the invention, or whose severity may be attenuated, include, without limitation, Acute Respiratory Distress Syndrome, Systemic Inflammatory Response Syndrome, secondary or nosocomial infections, Multiple Organ Failure, and Acute Lung Injury, including death caused by one or more of these syndromes.

In treating sepsis, the treatment may begin after the infection process has cleared sufficiently so that the risk to the patient arises more from inflammation-mediated complications than from the primary infection. Prior to this, the primary treatment can be with an appropriate antimicrobial agent.

Patients who may benefit by use of the methods of the present invention include, without limitation, patients who have suffered from blunt trauma and/or penetrating trauma. Blunt trauma includes blunt injuries, such as, e.g., those caused by traffic accidents or falls, which could result in one or more of liver injuries, multiple fractures, brain contusions, as well as lacerations of the spleen, lungs, or diaphragm. Blunt trauma is generally accompanied by more extensive tissue damage as compared to penetrating trauma and, consequently, more small vessel bleeding. Penetrating trauma includes penetrating injuries, such as, e.g., those caused by gun shot wounds or stab wounds, which could result in penetration of the inferior vena cava, liver damage, lung injury, injury to prostate, urinary bladder, thorax and liver lacerations, and wounds to the pelvis or chest.

Organ damage or organ failure encompass, without limitation, damage to the structure and/or damage to the functioning of the organ in kidney, lung, adrenal, liver, bowel, cardiovascular system, and/or haemostatic system. Examples of organ damage include, but are not limited to, morphological/structural damage and/or damage to the functioning of the organ such as, for example accumulation of proteins (for example surfactant) or fluids due to pulmonary clearance impairment or damage to the pulmonary change mechanisms or alveolo-capillary membrane damage. The terms “organ injury”, “organ damage” and “organ failure” may be used interchangeably. Normally, organ damage results in organ failure. By organ failure is meant a decrease in organ function compared to the mean, normal functioning of a corresponding organ in a normal, healthy person. The organ failure may be a minor decrease in function (e.g., 80-90% of normal) or it may be a major decrease in function (e.g., 10-20% of normal); the decrease may also be a complete failure of organ function. Organ failure includes, without limitation, decreased biological functioning (e.g., urine output), e.g., due to tissue necrosis, loss of glomeruli (kidney), fibrin deposition, haemorrhage, oedema, or inflammation. Organ damage includes, without limitation, tissue necrosis, loss of glomeruli (kidney), fibrin deposition, haemorrhage, edema, or inflammation.

Lung damage encompasses, but is not limited to, morphological/structural damage and/or damage to the functioning of the lung such as, for example accumulation of proteins (for example surfactant) or fluids due to pulmonary clearance impairment or damage to the pulmonary change mechanisms or alveolo-capillary membrane damage. The terms “lung injury”, “lung damage” and “lung failure” may be used interchangeably.

Methods for testing organ function and efficiency, and suitable biochemical or clinical parameters for such testing, are well known to the skilled clinician. Attenuation of organ failure or damage encompasses any improvement in organ function as measured by at least one of the well known markers of function of said organs

### Pharmaceutical Compositions

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants and the like.

Oral dosages can be established based on data such as provided below in the examples, typically projecting somewhat higher amounts for oral administration than for parenteral administration. Dosages may be administered for example 1 to 3 times per day. The exact dosage will depend upon the frequency and mode of administration, the sex, age, weight and general condition of the subject treated, the nature and severity of the condition treated and any concomitant diseases to be treated and other factors evident to those skilled in the art.

For parenteral routes such as intravenous, intrathecal, intramuscular and similar administration, typically doses are in the order of about half the dose employed for oral administration.

The compounds of this invention are generally utilized as the free substance or as a pharmaceutically acceptable salt thereof. One example is a base addition salt of a compound having the utility of a free acid. When a compound of the formula (I) contains a free acid such salts are prepared in a conventional-manner by treating a solution or suspension of a free acid of the formula (I) with a chemical equivalent of a pharmaceutically acceptable base.

For parenteral administration, solutions of compounds in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the novel compounds of the formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

The following examples further illustrate the present invention, but of course, should not be construed as in any way limiting its scope.

### EXAMPLE 1

### Blockage of S1P Synthesis Prior to Traumatic, Hemorrhagic Shock

A rat traumatic, hemorrhagic shock model was used (32). 4-[4-(4-Chloro-phenyl)-thiazol-2-ylamino]-phenol (SKI-2) (estimated plasma level 30 μM) or vehicle (DMSO) were given IP at the time of laparotomy, prior to shock. Animals were then underwent T/HS, were resuscitated with shed blood and sacrificed by exsanguination 3 hours after resuscitation. We assayed lung injury as % Evans Blue dye leak, which provides a measure of the leakiness of lung capillaries. PMN activation was assessed as CD11b expression by flow cytometry (Table 1).

| | Vehicle | SKI-2 |
|---|---|---|
| CD11b (MFI) | 322 ± 71 | 225 ± 18* |
| Evans Blue (%) | 12.5 ± 7.2 | 3.8 ± 1.7* |

Phlebotomy volumes were identical in the treatment and placebo groups after treatment with SKI-2. No unexpected hemodynamic effects were noted.

### EXAMPLE 2

### Blockage of SOCE Channels Prior to Traumatic, Hemorrhagic Shock

Identical attenuation of lung injury as in Example 1 was seen using N-propargyl-nitrendipine to inhibit S1P/SOC in pre-treatment and in each case, diminished lung PMN infiltration was confirmed by myeloperoxidase (“MPO”) assay (not shown). The amount of MRS-1845 administered IP was calculated to target a final concentration of 30 μm with the blood volume of the rat assumed to be 100 ml/kg Phlebotomy volumes were identical in the treatment and placebo groups after treatment with N-propargyl-nitrendipine (“MRS-1845”). No unexpected hemodynamic effects were noted.

### EXAMPLE 3

### Post Shock Treatments

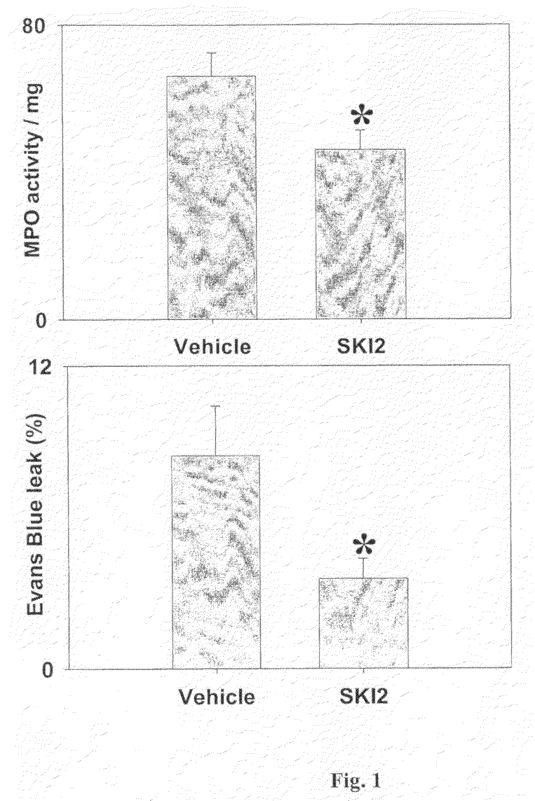

Male rats underwent standard T/HS. 4-[4-(4-Chloro-phenyl)-thiazol-2-ylamino]-phenol or N-propargyl-nitrendipine (estimated plasma level 30 μM for each) or vehicle were administered IP after the return of 10% of total shed blood to the animal. Different groups of animals (n=6/group) were sacrificed 3 hours after reperfusion for bronchoalveolar lavage and Evan's Blue dye leak assay, or to harvest the lungs for MPO assays (FIG. 1). Both 4-[4-(4-chloro-phenyl)-thiazol-2-ylamino]-phenol and N-propargyl-nitrendipine prevented PMN accumulation and subsequent capillary-alveolar leakage.

FIG. 1 shows that post-shock SOC treatment with 4-[4-(4-chloro-phenyl)-thiazol-2-ylamino]-phenol inhibits PMN mediated lung injury after T/HS as assessed either by pulmonary PMN infiltration (MPO assay) or by alveolar-capillary albumin leakage (EB dye). N=6 animals per condition, *p<0.01 for inter-group comparisons. Post-treatment with N-propargyl-nitrendipine yielded the same effects.

### EXAMPLE 4

### Post Shock Treatments Prevent PMN Priming

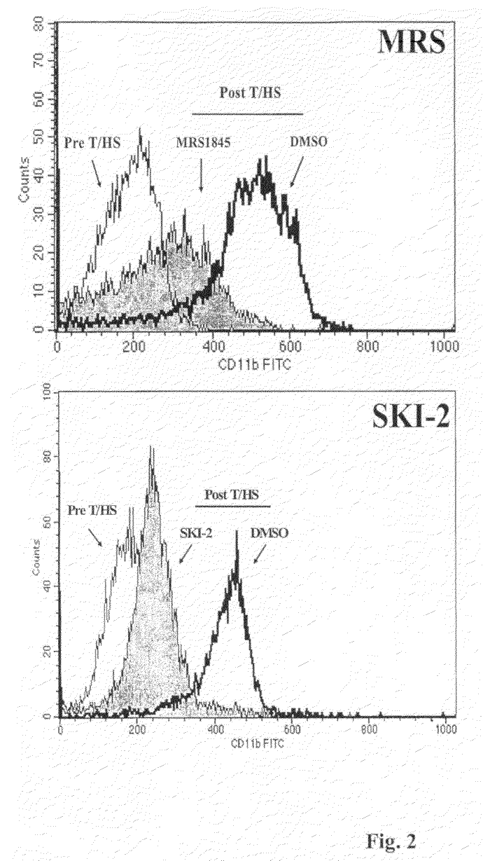

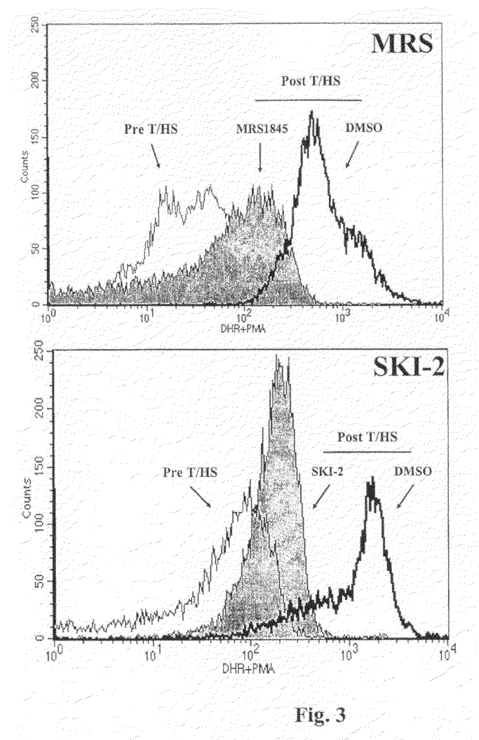

Male rats underwent T/HS with or without 4-[4-(4-chloro-phenyl)-thiazol-2-ylamino]-phenol or N-propargyl-nitrendipine administered IP in post-treatment as above. Small (200 μL) whole blood samples removed prior to shock and 3 hrs post reperfusion were assayed for PMN CD11b (FIG. 2) and respiratory burst (FIG. 3) by flow cytometry. FIG. 2 shows S1P/SOC inhibition in-vivo either by 4-[4-(4-chloro-phenyl)-thiazol-2-ylamino]-phenol or N-propargyl-nitrendipine given in post-treatment decreased CD11b expression almost to the level seen prior to shock. Left histogram, preshock; Right histogram, 3 h post-shock; Center (dark) histogram, 3 h post-shock PMN from animal treated with the indicated drug during resuscitation. Representative flow histograms, n=3-4 per condition. P<0.01, ANOVA/Tukey's test for all inter-group comparisons. FIG. 3 shows S1P/SOC inhibition in-vivo either by 4-[4-(4-chloro-phenyl)-thiazol-2-ylamino]-phenol or N-propargyl-nitrendipine given in post-treatment decreased PMN priming for respiratory burst initiated by PMA (phorbol 12-myristate 13-acetate) almost to the levels seen prior to shock. Representative flow histograms are arranged as in FIG. 2, n=3-4 per condition. P<0.01 by ANOVA/Tukey's test for all inter-group comparisons.

### EXAMPLE 5

### Post Shock Treatments Prevents Phosphorylation of PMN p38-MAP Kinase

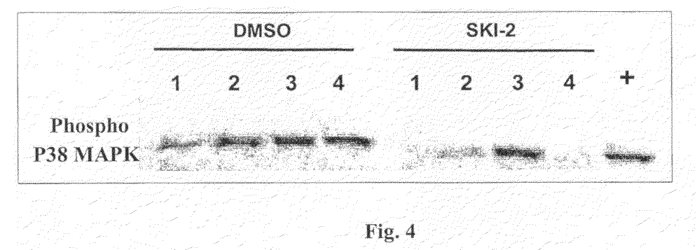

The phosphorylation of p38-mitogen associated protein kinase [“p38 MAP kinase”] in the presence of inflammatory stimuli is a key cellular mechanism involved in irreversible neutrophil activation and post-shock organ injury. Male rats underwent T/HS with or without 4-[4-(4-chloro-phenyl)-thiazol-2-ylamino]-phenol or N-propargyl-nitrendipine administered IP in post-treatment as above. At the end of 3 hours post reperfusion, animals were sacrificed by exsanguination. PMN were isolated. Whole cell lysates were probed using specific antibodies for phosphorylated p38 MAP kinase. SOC inhibition via either strategy led to diminished activation. The results of Western blots for phosphorylated p38 MAP kinase are shown in FIG. 4. Lysates of rat PMN were sampled 3 hours after T/HS. Animals were treated with 4-[4-(4-chloro-phenyl)-thiazol-2-ylamino]-phenol or vehicle (DMSO) at the time of resuscitation. Positive controls (+) were rat PMN activated in vitro with fMLP. PMN from treated rats showed attenuation of MAPK activation. Attenuation of p38 MAP Kinase phosphorylation and activation was also seen after N-propargyl-nitrendipine (not shown). Thus SIP/SOC inhibition diminishes PMN p38 MAP kinase activation by T/HS.

### EXAMPLE 6

### Hemodynamic Tolerance of Treatment

In all the above experiments, rats undergoing T/HS were treated with SKI-2 or N-propargyl-nitrendipine given as an IP bolus just after the BP nadir. At this point, MAP has risen only from 30-40 to approximately 50 mmHg. No episodes of hypotension were seen and all animals survived the acute shock phase.

### Additional Definitions

The following terms shall have, for the purposes of this application, the respective meanings set forth below.

Effective Amount

To treat the indications of the invention, an effective amount of a pharmaceutical compound will be recognized by clinicians but includes an amount effective to treat, reduce, alleviate, ameliorate, eliminate or prevent one or more symptoms of the disease sought to be treated or the condition sought to be avoided or treated, or to otherwise produce a clinically recognizable favorable change in the pathology of the disease or condition. Thus, an effective amount can be, for example, an amount that reduces the severity or duration of acute respiratory distress syndrome or acute lung injury. In combination treatments, as are likely for sepsis, it may be that a relatively lower amount nonetheless provides a clinical benefit.

Treatment

“Treatment” means the management and care of a patient for the purpose of combating a disease, disorder or condition. The term is intended to include the delaying of the progression of the disease, disorder or condition, the alleviation, amelioration or relief of symptoms and complications, and/or the cure or elimination of the disease, disorder or condition. The animal to be treated is preferably a mammal, in particular a human being.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

### BACKGROUND CITATIONS

1. MacKenzie, E. J., et al.: Acute hospital costs of trauma in the United States: implications for regionalized systems of care. J Trauma 30: 1096-101; discussion 1101-3 (1990).
2. Satcher, D.: From the surgeon general: injury: An overlooked global health concern. Jama 284: 950 (2000).
3. Bone, R. C., et al.: American College of Chest Physicians/Society of Critical Care Medicine Consensus Conference: Definitions for sepsis and organ failure and guidelines for the use of innovative therapies in sepsis. Crit. Care Med 20: 864-874 (1992).
4. Simms, H. H.: Polymorphonuclear leukocytes: their role as central cellular elements in shock pathogenesis. Shock 4: 225-31 (1995).
5. Simms, H. H., and D'Amico, R.: Polymorphonuclear leukocyte dysregulation during the systemic inflammatory response syndrome. Blood 83: 1398-407. (1994).
6. Rosenbloom, A. J., et al.: Leukocyte activation in the peripheral blood of patients with cirrhosis of the liver and SIRS. Correlation with serum interleukin-6 levels and organ dysfunction. Jama 274: 58-65. (1995).
7. Botha, A. J., et al.: Postinjury neutrophil priming and activation states: therapeutic challenges. Shock 3: 157-66. (1995).
8. Wagner, J. G., and Roth, R. A.: Neutrophil migration during endotoxemia. J Leukoc Biol 66: 10-24. (1999).
9. Hogg, J. C.: Neutrophil kinetics and lung injury. Physiol Rev 67: 1249-95 (1987).
10. Partrick, D. A., et al.: Neutrophil priming and activation in the pathogenesis of postinjury multiple organ failure. New Horiz 4: 194-210 (1996).
11. Yao, Y. M., et al.: The inflammatory basis of trauma/shock-associated multiple organ failure. Inflamm Res 47: 201-10. (1998).
12. Naka, Y., et al.: Failure to express the P-selectin gene or P-selectin blockade confers early pulmonary protection after lung ischemia or transplantation. Proc Natl Acad Sci USA 94: 757-61. (1997).
13. Nagase, T., et al.: Acute lung injury by sepsis and acid aspiration: a key role for cytosolic phospholipase A2. Nat Immunol 1: 42-6. (2000).
14. ARDS, N. P. o.: Conference report: Mechanisms of acute respiratory failure. Am Rev Respir Dis 115: 1071-8. (1977).
15. Brower, R. G.: Ventilation with lower tidal volumes as compared with traditional tidal volumes for acute lung injury and the acute respiratory distress syndrome. The Acute Respiratory Distress Syndrome Network. N Engl J Med 342: 1301-8. (2000).
16. Adams, J. M., et al.: Early trauma PMN responses to chemokines are associated with development of sepsis, pneumonia and organ failure. J Trauma 51: 452-7. (2001).
17. Hauser, C. J., et al.: Integrated stimulation by CXC chemokines enhances PMN [Ca2+]i signaling in trauma and ARDS. Surgery 126: 208-15 (1999).
18. Hauser, C. J., et al.: Priming of neutrophil [Ca2+]i signaling and oxidative burst by human fracture fluids. J Trauma 47: 854-8 (1999).
19. Hauser, C. J., et al.: Major trauma enhances store-operated calcium influx in human neutrophils. J Trauma 48: 592-7 (2000).
20. Adams, J. M., et al.: Damage control abdominal packing causes prolonged suppression of neutrophil IL-8 responses and phagocytosis. In Surgical Forum, vol. 51. American College of Surgeons, Chicago, Ill., 2000, pp. 178-180.
21. Adams, J. M., et al.: G-protein receptor responses in trauma neutrophils. J Trauma 49: 1096-101 (2000).
22. Adams, J. M., et al.: The immunomodulatory effects of damage control abdominal packing on local and systemic neutrophil activity. J Trauma 50: 792-800. (2001).
23. Nusse, O., et al.: Store-operated Ca2+ influx and stimulation of exocytosis in HL-60 granulocytes. J Biol Chem 272: 28360-7 (1997).
24. Bei, L., et al.: Extracellular Ca2+ regulates the respiratory burst of human neutrophils. Biochim Biophys Acta 1404: 475-83 (1998).
25. Dolmetsch, R. E., et al.: Differential activation of transcription factors induced by Ca2+ response amplitude and duration. Nature 386: 855-8 (1997).
26. Whyte, M. K., et al.: Transient elevations of cytosolic free calcium retard subsequent apoptosis in neutrophils in vitro. J Clin Invest 92: 446-55 (1993).
27. Abraham, E.: Neutrophils and acute lung injury. Crit. Care Med 31: S195-9. (2003).
28. Pober, J. S., and Cotran, R. S.: Cytokines and endothelial cell biology. Physiol Rev 70: 427-51 (1990).
29. Luster, A. D.: Chemokines-chemotactic cytokines that mediate inflammation. N Engl J Med 338: 436-45 (1998).
30. Itagaki, K., and Hauser, C. J.: Sphingosine 1-phosphate, a diffusible calcium influx factor mediating store-operated calcium entry. J Biol Chem 278: 27540-7 (2003).
31. Adams, Hauser et al J. Trauma 51(3):452-6 (2001).
32. Adams, C. A., Jr., et al.: Trauma-hemorrhage-induced neutrophil priming is prevented by mesenteric lymph duct ligation. Shock 18: 513-7 (2002).

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of treating trauma or sepsis comprising:
administering an effective amount of one or both of a SOC channel-selective inhibitor or a SphK inhibitor to an animal at risk of inflammation-mediated organ damage from trauma or sepsis.

2. The method of claim 1, wherein the animal is at risk of inflammation-mediated organ damage from trauma.

3. The method of claim 1, wherein the animal is at risk of inflammation-mediated organ damage from sepsis.

4. The method of claim 4, further comprising:
administering an effective amount of an antimicrobial agent in a dosage regimen effective to provide a clinical improvement in the sepsis and thereafter beginning said administering one or both of a SOC channel-selective inhibitor or a SphK inhibitor.

5. The method of claim 1, wherein said inflammation-mediated organ damage is Acute Respiratory Distress Syndrome, Systemic Inflammatory Response Syndrome, Multiple Organ Failure, or Acute Lung Injury.
